(19) United States
(12) Patent Application Publication
(10) Pub. No.: US 2022/0202548 A1
(43) Pub. Date: Jun. 30, 2022

(54) AN APPARATUS FOR TRACKING AND RECORDING THE MOVEMENTS OF A PERSON'S JAW AND THE RELATIVE METHOD

(71) Applicants: Giuseppe RAMPULLA, Tizzano Val Parma (PR) (IT); Luca PIGHIN, Zoppola (PN) (IT)

(72) Inventors: Giuseppe RAMPULLA, Tizzano Val Parma (PR) (IT); Luca PIGHIN, Zoppola (PN) (IT); Enrico FERMI, PIACENZA (IT); Diego CROVATO, SAONARA (PD) (IT)

(21) Appl. No.: 17/608,048

(22) PCT Filed: Apr. 30, 2020
(86) PCT No.: PCT/IB2020/054087

§ 371 Date: Nov. 1, 2021

(30) Foreign Application Priority Data
May 2, 2019 (IT) 102019000006498

(51) Int. Cl. A61C 19/045; A61B 90/00; A61B 5/00

(52) U.S. Cl. CPC A61C 19/045; A61B 90/39; A61B 2090/397; A61B 5/0088; A61B 2090/3912; A61B 5/0077

(57) ABSTRACT

Disclosed is an apparatus for tracking and recording the movements of a person's jaw including a reference system with two parts to be fixed to the respective dental arches, an infra-red light projector, a video camera sensitive to infra-red light and a computerized system connected to the projector and to the video camera, wherein the projector projects patterns of points of light onto the face and onto the reference system and wherein the computerized system includes a processing unit configured to process the images taken by the video camera to calculate, for each of these, the distance of at least the points of light that are on the surface of the markers with respect to a pre-determined point with known coordinates, by way of a comparison of the image of the projected pattern with an image of a reference pattern.

### BACKGROUND OF THE INVENTION

### Field of the Invention

The present invention concerns an apparatus for tracking, reconstructing and recording the movements of a person's jaw during mastication. In particular, the invention concerns an apparatus able to detect and reconstruct the aforesaid movements in a three-dimensional system.

### Description of the Related Art

The study of jaw movement and its reconstruction/simulation are particularly useful both for the dentist and for the dental technician. A simple, but rather inaccurate, apparatus used to reconstruct the movements of a jaw is the articulator. The casts of patient's dental arches are positioned in this device, which reproduces the basic opening and closing movement of the jaw. These devices can be used by the dental technician to produce rehabilitation medical devices, such as bridges, mouth splints or the like, which are in turn used by the dentist for the functional or esthetic restoration of a patient's teeth.

With only morphological data of the patient's teeth available, i.e., without information on the occlusal and masticatory function, it is not possible to faithfully replicate the movement of the person's mouth with these articulators. The main limit of apparatus of this kind is in fact the lack of information relating to the kinematics of the masticatory movement of the temporomandibular joint.

With these known apparatus the dentist must therefore subsequently adapt the rehabilitation devices prepared by the dental technician to the patient's actual occlusal condition. This implies an increase in costs and the risk of not obtaining a state-of-the-art rehabilitation device.

For these reasons various methods and related apparatus have been developed to track the movements of a person's jaw, in particular mastication, in order to obtain a digital model thereof that can be used to verify the correct shape and size of the rehabilitation device, for example a dental prosthesis.

Prior art systems are substantially based on three different technologies, namely “motion capture”, which uses video cameras and illuminators/projectors, inertial measurements, obtained through the use of accelerometers, gyroscopes or similar devices, and electromagnetic measurements, obtained with magnetic or inductive sensors, etc.

The present invention concerns the first type of apparatus, i.e. those that make use of optical sensors. Among these systems it is possible to further distinguish between systems of “marker based motion capture” type and those of “markerless” type. In the first case, stereo-photogrammetric techniques are used, which allow identification of the three-dimensional position and related movements of markers positioned on specific points of the patient. Within the sphere of analysis of jaw kinematics the aforesaid technique is used, taking as reference the “mandible-maxilla” region in order to obtain jaw kinematics.

Examples of prior art methods and related apparatus for tracking and reconstructing the masticatory movements of a person's jaw are described, for example, in US 2016/0262711 A1, U.S. Pat. No. 5,340,309 A and WO 2018115576 A1.

However, these prior art systems can be improved. In particular, some of them provide somewhat inaccurate data, for example due to the way in which the markers are fixed to the person's jaw or temporal or maxillary region. Moreover, prior art apparatus are often particularly complex and, besides being very costly, are also complicated to use. Finally, some of the prior art systems are also somewhat invasive and cause discomfort to the patient subjected to analysis.

For these reasons, jaw kinematics analysis applications implemented with optical systems are still relatively limited and not widely developed.

### SUMMARY OF THE INVENTION

In this context, the object of the present invention is to solve the limits of the prior art methods and apparatus.

In particular, the object of the invention is to propose an apparatus for tracking the movements of a person's jaw that is simple and practical for an operator to use, but at the same time provides accurate results.

A further object of the present invention is to provide an apparatus of this kind that is simple to use and relatively inexpensive to manufacture.

Another object of the invention is to provide an apparatus for tracking the movements of the jaw that does not cause discomfort to the patient being examined.

These and other objects are achieved with an apparatus that comprises:
a reference system comprising two parts to be attached to the person's upper and lower dental arches, each part including markers positioned in front of the face;
an infra-red light projector;
a video camera sensitive to infra-red light, configured to frame a scene that includes at least said reference system; and
a computerized system connected to the projector and to the video camera.

According to the invention, the infra-red light projector is placed in front of the person. Said projector is configured to project towards the person, in an area that includes at least the reference system, and preferably also the whole face, infra-red light beams that generate on the illuminated surfaces a pattern of points of light. The video camera is configured to record with a given frequency the projected images of the afore-mentioned pattern of points of light, at least on the reference system. These images can be recorded with the jaw still or moving, for example with a masticatory movement.

The computerized system comprises a processing unit configured to receive the images captured by the infra-red video camera and to calculate, for each image, the distance of at least the points of light that are on the surface of the markers with respect to a pre-determined point with known coordinates. Calculation of the distance takes place by means of a comparison of the image of the actual projected pattern with an image of a reference pattern. Said reference pattern, for example, can be provided by projecting the same light beams on a flat surface, parallel to the plane of the video camera and positioned at a known distance therefrom.

In fact, when the light beams projected by the projector encounter the surfaces of the markers and of the person's face, a variation of the distance between one point and the other is created based on the angle of the surfaces, as the video camera records a two-dimensional projection of the actual (three-dimensional) distribution of said points of light.

Therefore, for each of the points projected it is possible to calculate the distance from the known base point, generally coincident with the sensor of the video camera, triangulating its position with the one it would have in the reference pattern.

The apparatus thus configured is therefore able to directly determine the position in space of the markers, and hence to reconstruct the jaw kinematics in the three dimensions.

Moreover, the measurements to be carried out with this apparatus are not affected by any movements of the person's head while taking the images, as measurement of the distance of the markers is carried out with respect to a known reference system.

The computerized system preferably also includes a storage unit for storing at least the images taken by the video camera and the data calculated by the processing unit.

According to a preferred variant, the video camera is an RGB video camera sensitive to infra-red wavelengths. This makes it possible to record color images that, in the subsequent processing step, better described below, allow more precise recognition of the various elements framed, hence of the surfaces of the markers.

According to an aspect of the invention, each part of the reference system comprises three markers. Said markers preferably have a curved, and more preferably hemispherical, surface. In this way, in the various positions that the marker can take during the movement of the person's jaw or also skull, the surface of the marker always has areas at a different distance (depth) with respect to the video camera. This makes measurement of the distance of the various points of the pattern more accurate and hence gives an accurate reconstruction of the movements in the three dimensions.

According to a preferred embodiment, the markers are mounted on a rigid support and are positioned at the vertices of a triangle. Said triangle is preferably an isosceles triangle and is arranged with its height substantially vertical.

When the reference system is applied to the person, the lower part, i.e. the part attached to the lower dental arch and the jaw, has the apex of the aforesaid triangle facing upwards while the upper part of the reference system, i.e., the part attached to the upper dental arch, has the apex facing downwards, i.e., with said triangle overturned.

This substantially arrow-shaped positioning of the marker allows precise and immediate evaluation of the lateral movements of the mandible with respect to the maxilla.

Advantageously, the supports and the relative markers of the upper and lower part are identical in terms of shape and size so as to optimize both production and procurement costs for users.

According to an aspect of the invention said support comprises a plate made of an opaque material, preferably of a uniform color; typically said support is made of metal or a polymer material.

In this way, the video camera is prevented from recording the part of the person's face immediately behind the markers reducing possible disturbance effects. To this end, the uniform color of the support contributes, making the markers more distinguishable. Preferably, the outer surface of the markers also consists of or is coated with a reflecting material.

The markers have a diameter typically ranging from 5 mm to 20 mm, to offer an area that can be hit by a number of infra-red light beams sufficiently to obtain accurate calculation of the position of the marker.

The distance between the markers of one part, i.e., the length of the sides of the triangle, typically ranges from 30 mm to 60 mm.

According to an aspect of the present invention, said supports are each carried by a rigid supporting element that can be fixed directly to the upper and lower dental arch.

This type of connection makes it possible to reduce measurement errors to a minimum as the markers move substantially rigidly attached to the mandible and the maxilla.

Said supporting element comprises a fastening plate, to be fixed to the teeth by means of an adhesive product, such as resin or the like, and a rod that extends from the fastening plate and carries said support at the end. The rod preferably has a shape or orientation such that when the person's jaw is clamped, the vertices of the two supports of the lower and upper parts are close but do not touch each other.

To avoid change in the person's occlusal function, and hence natural jaw movement, the fastening plates are preferably structured so as to be fixed to the front surface of the teeth.

The method for tracking and recording the movements of a person's jaw according to the invention thus comprises at least the following steps:
providing at least one infra-red projector and at least one video camera sensitive to infra-red wavelengths;
providing a reference system comprising two parts, where each part comprises markers;
fixing the two parts of the reference system to a person's upper and lower dental arches, respectively, in such a way that the markers are positioned in front of the face;
positioning the person in front of the video camera and the infra-red projector;
projecting towards the person's face infra-red light beams adapted to form a pattern of points of light, and at the same time to record with a certain frequency images of a scene that includes at least the reference system or the whole face of the person onto which the pattern of points of light is projected;
processing the images taken with a computerized system configured to compare each image with an image of a reference pattern and to calculate the distance of at least the points of light projected onto the surface of the markers with respect to a pre-determined point with known coordinates.

More in detail, the step of processing the images includes calculating the position of the center of gravity of each marker and tracking their position movements in space. The position of the center of gravity of the various markers, in the preferred variant three in number for each part of the reference system and arranged at the vertices of an isosceles triangle, allows calculation of the center of each part of the reference system.

To improve recognition of the marker with respect to the rest of the scene recorded, the analysis method involves thresholding the brightness levels of the image and simultaneously the color levels.

At this point, subsequent processing of the image allows calculation of the three-dimensional coordinates (X, Y, Z) of the marker, instant by instant, as a function of the acquisition frequency of the video camera.

The method according to the invention involves analysis of the kinematics of the temporomandibular joint both in static position (occlusion) and in movement, for example during mastication.

The kinematic analysis thus obtained is able to provide position, angles and rototranslations of the reference system attached to the jaw and hence comprises all the kinematic information required and sufficient for replication of the movement.

The data provided can be interfaced with a CAD/CAM system and comprise the three coordinates of position and orientation by means of the three Euler angles of both parts of the reference system attached to the person's mandible and maxilla, as well as the position data relating to one part of the reference system with respect to the other (three position and three rotation coordinates).

### BRIEF DESCRIPTION OF THE DRAWINGS

Further characteristics and details of the invention will be more apparent from the following description, provided by way of non-limiting example, and from the accompanying drawings, wherein:

FIG. 1 is a perspective view of the reference system of the apparatus according to the invention;

FIG. 2 is a perspective view of the reference system applied to a person's dental arches;

FIG. 3 is a schematic view of the apparatus according to the invention in the condition of use.

### DESCRIPTION OF THE PREFERRED EMBODIMENTS

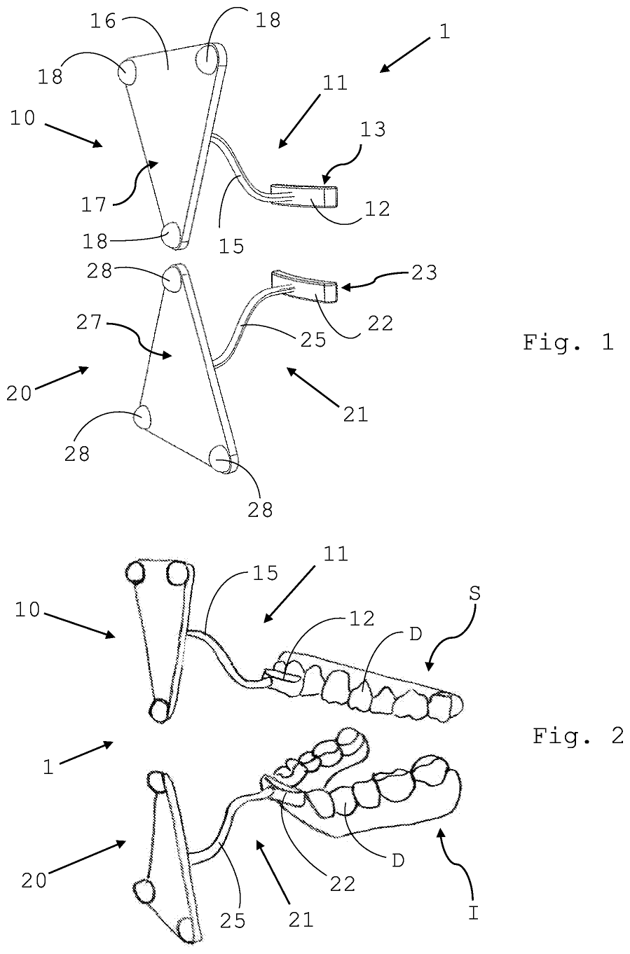

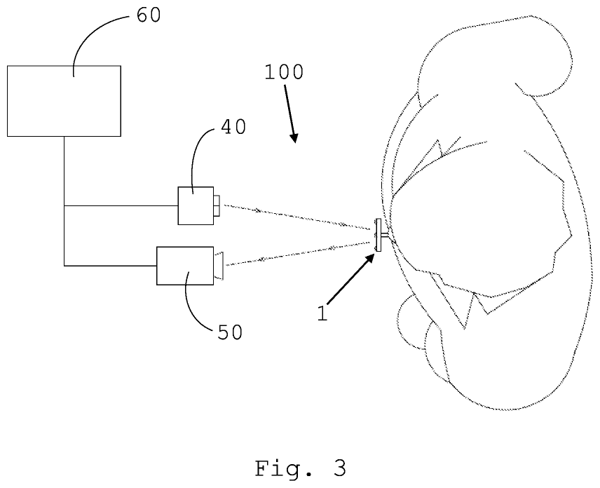

With reference to FIG. 1, this illustrates the reference system 1 of the apparatus according to the invention. The reference system comprises two parts 10, 20 to be attached to the upper and lower dental arch, respectively. Each part comprises a supporting element 11, 21 with a curved fastening plate 12, 22 that has a concave supporting surface 13, 23 adapted to be placed against the front surfaces of the teeth D, as shown in FIG. 2. Fixing of the plate 12, 22 typically takes place by means of gluing, although alternative fixing means, for example of mechanical type, would also be possible.

A curved rod 15, 25, and more precisely curved in a substantially vertical plane, extends from the fastening plate 12, 22. A support 16, 26 comprising a plate in the shape of isosceles triangle is connected to the end of the rod. Preferably, the plate and the rod, which are typically disposable, are connected movably to the support so that the latter can be re-utilized several times for the same person or for different people.

Three hemispherical markers 18, 28 are applied to the vertices of the triangle, on the front surface 17, 27 of the support 16. 26. Said markers have a reflective surface, for example coated with aluminum powder or with a colored reflective paint, preferably of a uniform color.

FIG. 3 schematically illustrates the apparatus 100 according to the present invention in an example of use.

In the example in the figure, the reference system 1 is applied to the teeth of a person P whose jaw kinematics require to be tracked. The apparatus 100 further comprises a projector 40 adapted to project infra-red light beams according to a pseudo-random pattern and a video camera 50 of RGB type sensitive to infra-red wavelengths. The video camera 50 and the projector 40 are positioned side by side, facing towards the person's face, as shown in FIG. 3.

The projector 40 and the video camera 50 are connected to a computerized system 60 adapted to receive and process the images taken by the video camera.

The computerized system typically comprises at least a processing unit, a storage unit and preferably also a video interface.

The computerized system is further equipped with a computer program that allows the processing unit to process the images taken to obtain a three-dimensional digital reconstruction of the movement of the person's jaw.

The method implemented through said computerized system involves applying a selective filter to the images to search, in succession, for the greatest brightness and color levels.

Unlike prior art methods commonly used in movement analysis, which are based on calculation of the 2D centers of gravity with subsequent calculation of the three-dimensional coordinates, by means of triangulation, in the present invention three-dimensional data are processed directly.

In detail, the analysis involves the processing of clouds of three-dimensional points of the scene illuminated with the infra-red light beams and recorded by the video camera. The pattern of light beams projected is of a pseudo-random nature and makes it possible to obtain a set of 3D points of coordinates X, Y, Z, which form the hemispherical surfaces of the markers.

The processing method of the images then involves applying a filter to all the 3D points by means of statistical operators to eliminate those that do not satisfy certain criteria.

In particular, the distribution of the distances between the nearby points is calculated and more precisely for each point the average distance between it and all its nearby points is calculated. Assuming that the resulting distribution is of Gaussian type with a mean and standard deviation, all the points whose mean distances are outside a range defined by the global mean of the distances and by the standard deviation are considered as abnormal values and hence removed from the set of data.

Euclidean clustering analysis is applied to the remaining data, i.e., the data are grouped into sub-groups, or clusters, which comprise the points with mathematical properties “similar” to one another, specifically those belonging to the hemispherical surfaces of the markers. This algorithm makes it possible to identify the clusters in which the elements belonging to them are more “similar” to one another with respect to the elements belonging to other sub-groups. The measurement of similarity is a function of the Euclidean distance between the objects and allows the construction of spherical clusters with similar sizes and densities. These spherical clusters correspond to the markers of the reference system.

The subsequent processing is the reconstruction of the effective spherical shape, for example by means of the MLS (Moving Least Squares) technique, which performs smoothing and resampling filtering of the data, to eliminate any discontinuities.

However, the distances calculated and used in the preceding step can, in some cases, be affected by small errors that lead to irregularities in the data that are difficult to eliminate with the statistical method alone. To overcome this, a resampling is preferably carried out to attempt to recreate the missing parts of the surface of the marker by means of polynomial interpolation of higher degree between the surrounding data points.

In the scientific literature, processes for reconstructing surfaces based on polynomial approximation methods (least squares approximation) are frequently described, but not to obtain interpolated data. Therefore, in the analysis method, the MLS Moving Least Squares approximation variant, associated with appropriate weights, is implemented.

At this point, the centers of these hemispheres are calculated by means of an algorithm for estimation of the parameters of the spherical surface and for estimation of the normal directions of each point.

Once the centers of the markers of the two parts of the reference system applied to the person have been defined, the center of the Cartesian coordinate system attached to the lower dental arch and to the upper dental arch is calculated.

The systems are referred with respect to the movement space of the patient and can provide data relating to the movement between them.

With the algorithm implemented in the present invention, measuring as reference parameter a distance between two markers, a precision in the order of units per thousand is obtained.

The invention has been described purely for non-limiting illustrative purposes, according to some preferred embodiments. The person skilled in the art may make many other embodiments and variants, all falling within the scope of protection of the appended claims.

1. An apparatus for tracking and recording the movements of a person's jaw, comprising:
a reference system comprising two parts to be attached to the person's upper and lower dental arches, respectively, each part including markers positioned in front of the face;
an infra-red light projector;
a video camera sensitive to infra-red light, configured to frame a scene that includes at least said reference system; and
a computerized system connected to the projector and to the video camera;
wherein said projector is configured to project towards the person, in an area that includes at least the reference system, infra-red light beams that generate on the illuminated surfaces a pattern of points of light, wherein the video camera is configured to record with a given frequency the projected images of the afore-mentioned pattern of points of light, at least on the markers of the reference system, and wherein said computerized system comprises:
a processing unit configured to receive the images taken by the infra-red video camera and to calculate, for each image, the distance of at least the points of light that are on the surface of the markers with respect to a pre-determined point with known coordinates, by means of a comparison of the image of the projected pattern with an image of a reference pattern;
a storage unit for storing at least the images taken by the video camera and the data calculated by the processing unit.

2. The apparatus according to claim 1, wherein generation of the infra-red light beams is of a pseudo-random nature.

3. The apparatus 100) according to claim 1, wherein each part of the reference system comprises three hemispherical markers.

4. The apparatus according to claim 1, wherein the markers have an outer surface consisting of or coated with a reflecting material.

5. The apparatus according to claim 1, wherein the markers are mounted on a rigid support and are positioned at the vertices of an isosceles triangle arranged with its the isosceles triangle's height substantially vertical.

6. The apparatus according to claim 5, wherein, when the reference system is applied to the person, the lower part has the apex of the triangle turned upwards while the upper part of the reference system has the apex turned downwards.

7. The apparatus according to claim 5, wherein the supports and relative markers of the upper and lower parts are identical in terms of shape and size.

8. The apparatus according to claim 5, wherein said support comprises a plate made of an opaque material or a material of a uniform color.

9. The apparatus according to claim 5, wherein said supports are each carried by a rigid supporting element that can be directly fixed to the upper or lower dental arch, respectively.

10. The apparatus according to claim 9, wherein said supporting element comprises a fastening plate, to be fixed to the teeth by means of an adhesive product or by mechanical means, and a rod j (15, 25) that extends from the fastening plate and carries the support at the end.

11. A method for tracking and recording the movements of a person's jaw comprising at least the following steps:
providing at least one infra-red projector and at least one video camera sensitive to infra-red wavelengths;
providing a reference system comprising two parts, where each part comprises markers;
fixing the two parts of the reference system to a person's upper and lower dental arches, respectively, in such a way that the markers are positioned in front of the face;
positioning the person in front of the video camera and the infra-red projector;
projecting towards the person's face infra-red light beams adapted to form a pattern of points of light, and at the same time to record with a certain frequency images of a scene that includes at least the reference system or the whole face of the person onto which the pattern of points of light is projected;
processing the images taken in real time with a computerized system configured to compare each image with an image of a reference pattern and to calculate the distance of at least the points of light projected onto the surface of the markers with respect to a pre-determined point with known coordinates.

12. The apparatus 100) according to claim 1, wherein each part of the reference system comprises three hemispherical markers.

13. The apparatus according to claim 2, wherein the markers have an outer surface consisting of or coated with a reflecting material.

14. The apparatus according to claim 3, wherein the markers have an outer surface consisting of or coated with a reflecting material.

15. The apparatus according to claim 2, wherein the markers are mounted on a rigid support and are positioned at the vertices of an isosceles triangle arranged with the isosceles triangle's height substantially vertical.

16. The apparatus according to claim 3, wherein the markers are mounted on a rigid support and are positioned at the vertices of an isosceles triangle arranged with the isosceles triangle's height substantially vertical.

17. The apparatus according to claim 4, wherein the markers are mounted on a rigid support and are positioned at the vertices of an isosceles triangle arranged with the isosceles triangle's height substantially vertical.

18. The apparatus according to claim 6, wherein the supports and relative markers of the upper and lower parts are identical in terms of shape and size.

19. The apparatus according to claim 6, wherein said support comprises a plate made of an opaque material or a material of a uniform color.

20. The apparatus according to claim 7, wherein said support comprises a plate made of an opaque material or a material of a uniform color.
